(19) United States
(12) Patent Application Publication
(10) Pub. No.: US 2018/0200315 A1
(43) Pub. Date: Jul. 19, 2018

(54) CANNABINOID AND CAFFEINE EMULSIFICATIONS

(71) Applicant: Ronald SILVER, New York, NY (US)

(72) Inventor: Ronald SILVER, New York, NY (US)

(21) Appl. No.: 15/565,136

(22) PCT Filed: Apr. 14, 2017
(86) PCT No.: PCT/US17/27543

§ 371 Date: Oct. 9, 2017

Related U.S. Application Data
(60) Provisional application No. 62/323,154, filed on Apr. 15, 2016.

(51) Int. Cl. A61K 36/185; A61K 9/107; A61K 31/522; A61K 47/40; A61K 47/46; A61K 31/352; A61K 47/36; A23G 1/48; A23L 33/105

(52) U.S. Cl. CPC A61K 36/185; A61K 9/107; A61K 31/522; A61K 47/40; A23V 2002/00; A61K 31/352; A61K 47/36; A23G 1/48; A23L 33/105; A61K 47/46

(57) ABSTRACT

A cannabinoid emulsification comprising at least one emulsifying agent; an aqueous vehicle; a base oil; cannabis oil; and caffeine; wherein the cannabinoid emulsification is bio-available, highly metabolizable and fast acting when ingested by the user.

### FIELD OF THE INVENTION

The present invention relates to cannabinoid and caffeine emulsifications that are bio-available, fast action and highly metabolizable.

### BACKGROUND OF THE INVENTION

Cannabinoids are chemical compounds found in the cannabis plant that interact with receptors in the brain and body to create various effects Herbal cannabis contains over 400 compounds including over 100 cannabinoids, which are aryl-substituted meroterpenes unique to the plant genus Cannabis. The pharmacology of most of the cannabinoids is largely unknown but the most potent psychoactive agent, Δ⁹-tetrahydrocannabinol (Δ⁹-THC, or THC), has been isolated, synthesized and much studied due to its abundance and psychoactive attributes. Other plant cannabinoids include Δ⁸-THC, cannabinol and cannabidiol (CBD). These and other cannabinoids have additive, synergistic or antagonistic effects with THC and may modify its actions when herbal cannabis is smoked.

The best studied cannabinoids include tetrahydrocannabinol (THC), cannabidiol (CBD) and cannabinol (CBN). These structures are shown below in FIG. 1. All cannabinoids derive from cannabigerol-type compounds and differ mainly in the way this precursor is cyclized. The classical cannabinoids are derived from their respective 2-carboxylic acids (2-COOH) by decarboxylation (catalyzed by heat, light, or alkaline conditions).

The isolation of THC came from an Israeli chemist by the name of Raphael Mechoulam. In 1964, Mechoulam isolated and synthesized THC from Lebanese hashish, marking the beginning of cannabis research that would lead to the discovery of many other cannabinoids, cannabinoid receptors throughout the body, and “endocannabinoids” the THC-like compounds the human body naturally produces to maintain stability and health.

CBD and THC levels tend to vary among different plants. Marijuana grown for recreational purposes often contains more THC than CBD. However, by using selective breeding techniques, cannabis breeders can create varieties with high levels of CBD and next to zero levels of THC.

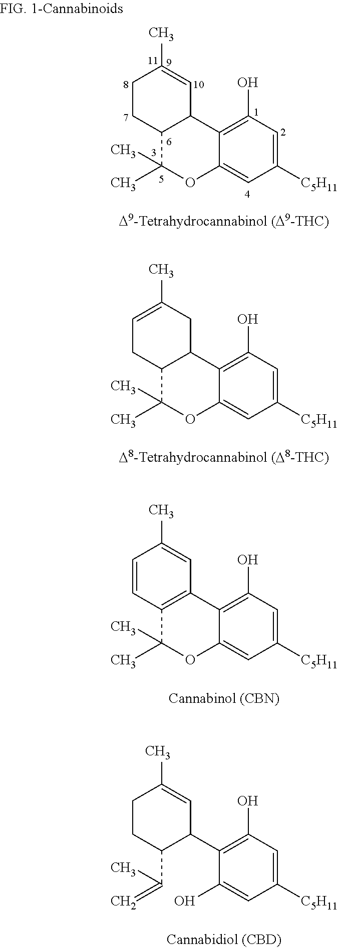

Humans and many other animals have receptor systems that THC binds to, and therefore can also reap the benefits of cannabinoids for both health and enjoyment. The endocannabinoid system (or “ECS”), is a group of specialized signaling chemicals, their receptors, and the metabolic enzymes that produce and break them down. These endocannabinoid chemical signals act on some of the same brain and immune cell receptors (CB1 and CB2) that plant cannabinoids like CBD and THC act on.

THC works by binding to cannabinoid receptors concentrated in the brain and central nervous system to produce psychoactive effects. The main difference between THC and CBD, both of which are very popular cannabinoids, is in their psychoactive effects. THC elicits strong cerebral euphoria, while CBD lacks psychoactive effects altogether. This basically comes down to the fact that THC activates CB1 receptors in the human brain while CBD does not.

It is well known that cannabinoids, especially CBD and THC have many medicinal benefits. CBD's subtle effects are primarily felt in pain, inflammation, and anxiety relief, as well as other medicinal benefits. CBD also does not have any adverse side effects that may occur with consumption of THC. Unlike THC, CBD also does not cause a high. While this makes CBD a significant advantage as a medicine, since health professionals prefer treatments with minimal side effects. CBD also appears to counteract the sleep-inducing effects of THC, which may explain why some strains of cannabis are known to increase alertness. CBD also acts to reduce the intoxicating effects of THC, such as memory impairment and paranoia.

THC has a wide range of short-term effects which may or may not be experienced depending on the individual and their body chemistry. Some positive short-term effects of THC include: elation, relaxation, sedation, pain relief, energy, hunger, drowsiness, slowed perception of time and laughter.

There are a variety of medical conditions for which THC offers benefits. The conditions include Post Traumatic Stress Disorder, neuropathic and chronic pain, insomnia, nausea, inflammation, arthritis, migraines, Cancer, Crohn's disease, fibromyalgia, Alzheimer's disease, Multiple sclerosis, Glaucoma, Attention deficit hyperactivity disorder (“ADHD”), sleep apnea and appetite loss.

Both CBD and THC have been found to present no risk of lethal overdose. However, to reduce potential side effects, medical users are better off using cannabis with higher levels of CBD.

Today the most common way to consume THC is through smoking although they can be consumed orally. However, known methods for orally administered THC have reduced bioavailablity due to low absorption and high first-pass metabolism in the digestive system. Thus there is a need for aqueous cannabinoid solutions.

Decarboxylation of the THC occurs with heating and is the key to enjoying THC, whether it is consumed by smoking or ingesting. In its raw form, cannabis is non-psychoactive, with its primary cannabinoid being THCA. However, by applying heat, either when lighting it in a pipe or cooking it into oil, the THCA is converted to THC.

The invention method provides an advantageous alternative to smoking cannabis by providing a water-soluble cannabinoid composition for oral ingestion that is bioavailable, highly metabolizable and fast acting.

As an aromatic terpenoid, THC has a very low solubility in water, but good solubility in most organic solvents, specifically lipids and alcohols.

The problem with edible cannabis products is they take a varied amount of time to take effect due to the liver's varied ability to process the THC molecule. Depending on liver function at the time, between 2-6% of the THC is able to be metabolized. This process makes it so the THC is absorbed in the esophagus and soft tissues, making it faster acting and more highly metabolizable.

U.S. Pat. No. 8,906,429 to Kolsky discloses lozenges made with THC, coconut oil, sugar and other ingredients. However, there is no use of emulsifiers, which is the main component that makes the cannabis oil hydrophilic and soluble in water.

The internet reference “I Drank Cannabis Coffee with Seattle Baristas” discloses coffee infused with cannabis in coconut oil and butter.

Unlike anything currently known, the purpose of the invention is to provide a cannabinoid emulsification to create cannabis oil infused products for medical and recreational use that are bioavailable, fast acting and highly metabolized, with consistent results that take place in a consistent amount of time.

The purpose of the invention is to provide a method to make cannabis oil water soluble using a combination of emulsifiers and variations in time and temperature of the reaction steps. The process results in a cannabinoid emulsification which can be used in a variety of edible products providing fast acting, bioavailability and highly metabolizable delivery of the cannabis oil.

Another purpose of the invention is to provide a line of cannabis oil and caffeine edible products that share a base of coconut water infused with coconut fat and to sell the products to wholesale distributors for retail sale in legal dispensaries.

Another purpose of the invention is to provide a line of cannabis oil and caffeine infused sugars and elixirs that share a base of coconut water infused with coconut fat and to sell the products to wholesale distributors for retail sale in legal dispensaries.

More specifically a purpose of the invention is to use the water soluble cannabinoid emulsification to treat Post Traumatic Stress Disorder, neuropathic and chronic pain, insomnia, nausea, inflammation, arthritis, migranes, Cancer, Crohn's disease, fibromyalgia, Alzheimer's disease, Multiple sclerosis, Glaucoma, Attention deficit hyperactivity disorder (“ADHD”), sleep apnea and appetite loss.

Yet another purpose of the invention emulsification is to treat pain, inflammation, and anxiety relief.

More specifically, the emulsification can be combined with chocolate and/or liquor to create edible products.

Yet another purpose of the invention composition is that it can be used to produce other edible products at home or professionally with predictable results, including being fast-acting, and highly metabolizable, which are referred to herein as super-charged. This lets consumers know that the invention products have markedly different results than other products. These compositions can be marketed in many forms, both in retail and wholesale manufacturing, as well as aiding companies with quality products to use for research and development.

### SUMMARY OF THE INVENTION

In the present invention, these purposes, as well as others which will be apparent, are achieved generally by a cannabinoid emulsification made of at least one emulsifying agent; an aqueous vehicle; a base oil; cannabis oil; and caffeine. The resulting cannabinoid emulsification is bio-available, highly metabolizable and fast acting when ingested by the user.

The aqueous vehicle is selected from the group consisting of coconut water, fruit juice, milk and water. The aqueous vehicle is in the range of 60% to 99.9% of the emulsification. The preferred vehicle is coconut water.

The base oil is selected from the group consisting of vegetable glycerine, almond oil, avocado oil, canola oil, coconut oil, corn oil, cottonseed oil, grapeseed oil, hazelnut oil, olive oil, extra virgin olive oil, palm oil, peanut oil, palm seed oil, pumpkin seed oil, safflower oil, sesame oil, soy oil, sunflower oil, vegetable oil and walnut oil and any oil high in saturated fats. The base oil is preferably in the range of 0.1% to 40% of the emulsification.

The cannabis oil is selected from the group consisting of tetrahydrocanniabinol (THC), cannabidiol (CBD) and other cannabinoid oils isolated from the marijuana plant.

The emulsifying agent is in the range of 0.15% and 2% of the total volume of the emulsification and is selected from the group consisting of xantham gum, guar gum, cyclodextrin, lecithin, carrageen, monoglycerides, natural emulsifiers and organic emulsifiers that are safe for ingestion by humans.

In a preferred embodiment the emulsifying agent is a combination of at least two emulsifying agents. In a most preferred embodiment cyclodextrin is used in combination with at least one other emulsifying agent selected from the group consisting of xantham gum, guar gum, lecithin, carrageen, monoglycerides, natural emulsifiers and organic emulsifiers that are safe for ingestion by humans

The cannabis oil in the emulsification is in the range of 5 mg to 30 mg per 2 ounces of the emulsification.

Caffeine is present in the emulsification in the range of 10 to 300 mg per 2 ounces of the emulsification. The caffeine can be in anhydrous form.

In the emulsification the base oil:aqueous vehicle ratio is between 1 to 10 grams of base oil per 2 ounces of the emulsification.

The invention also provides a method for making cannabinoid emulsifications comprising the steps of heating a base oil, preferably coconut oil, to between 120 to 220 degrees F. Adding at least one emulsifying agent, caffeine and cannabis oil to an aqueous vehicle and adding to the heated coconut oil to create a mixture. Blending the mixture in a high speed machine, while holding the temperature between 120 to 220 degrees F. to emulsify the mixture and then adding caffeine to the mixture.

The emulsifying agents are added in an amount between 0.15% and 2% of the total volume of the mixture and are selected from the group consisting of xantham gum, guar gum, cyclodextrin, lecithin, carrageen, monoglycerides, natural emulsifiers and organic emulsifiers that are safe for ingestion by humans.

The hot mixture is blended at high speed for between 30 seconds and 2 minutes. The resulting cannabinoid emulsification is bio-available, highly metabolizable and fast acting when ingested by the user.

Other objects, features and advantages of the present invention will be apparent when the detailed description of the preferred embodiments of the invention is considered which should be construed in an illustrative and not limiting sense

### DETAILED DESCRIPTION OF THE INVENTION

The invention provides a unique emulsified combination of cannabis oil, caffeine, and a base oil and aqueous vehicle, which are respectively, preferably coconut oil, and coconut water. Coconut oil is one of the best sources of excellent fatty acids. Emulsified with coconut water, cannabis oil and caffeine provide a beneficial experience for people experiencing a variety of ailments: insomnia, muscle aches, anxiety, etc, or are in recovery from surgery, or in chemotherapy. The emulsification makes the cannabis oil molecules water soluble, by modification from its normal hydrophobic state into a hydrophilic (“water-loving”), which makes the cannabis oil bioavialable, faster acting, and more highly metabolizable.

Bioavailability refers to the degree to which food nutrients, in this invention—cannabis oil—are available for absorption and utilization in the body. Bioavailability typically applies to nutrients and drugs which pass through first-pass metabolism, i.e. orally consumed substances. Anything absorbed in the gut first passes through the liver before reaching the rest of the circulation, and both the gut and liver may metabolize it to some extent.

Metabolizable refers to the process of changing food/substances into a form that can be used by your body. To process and use substances brought into your body by metabolism

The cannabinoid emulsification of the invention is made of at least one emulsifying agent; an aqueous vehicle; a base oil; cannabis oil; and caffeine.

### Emulsifiers

Emulsions are produced by dispersing normally unmixable material into another by mixing, colloidal milling or homogenization. The surface-active qualities of emulsifiers of the invention make them effective emulsifying agents that reduce mixing time and maintain the stability of the dispersion.

The emulsifying agent in the invention is present in the range of 0.15% to 2% of the composition. At least one emulsifying agent is used in the invention process which is selected from the group consisting of xanthan gum, guar gum, cyclodextrin, lecithin, carrageen, monoglycerides, natural emulsifiers and organic emulsifiers that are safe for ingestion by humans. In preferred embodiments, the emulsifying agent is a combination of at least two different emulsifying agents.

Cannabis oil, including THC and CBD, are not water-soluble, so it needs to be “trapped” in something with dual polarity that is, a compound that reconciles the fact that water is polar and the cannaboid is not. The emulsifiers provide this. Once trapped in the compound, the THC has new de facto properties, like the ability to dissolve in water, distribute itself evenly, and stay suspended in the solution. It also displays increased bioavailability: while the same amount of cannabis oil in an edible can take up to two hours to reach the bloodstream, the effects of water soluble cannabis oil dissolved in water can be felt more acutely, in as little as 10 minutes.

It is known that cannaboids are soluble in fat. It is also known that only water soluble substances can pass the intestine membrane. Fat is itself not water soluble because it is like cannaboids, uncharged. Fat absorption into the membrane requires substances with a dipole character to build up vehicles which can connect at the outer surface with water (charged side) and at the inner surface with the fat and the THC (uncharged side).

The specific emulsifiers used in the invention are detailed below.

### Xanthan Gum

Xanthan gum, which is also called xanthene, has the chemical formula C₁₃H₁₀O. Its molecular weight is 182.22 grams/mol. FIG. 2 shows the chemical structure of xantham gum.

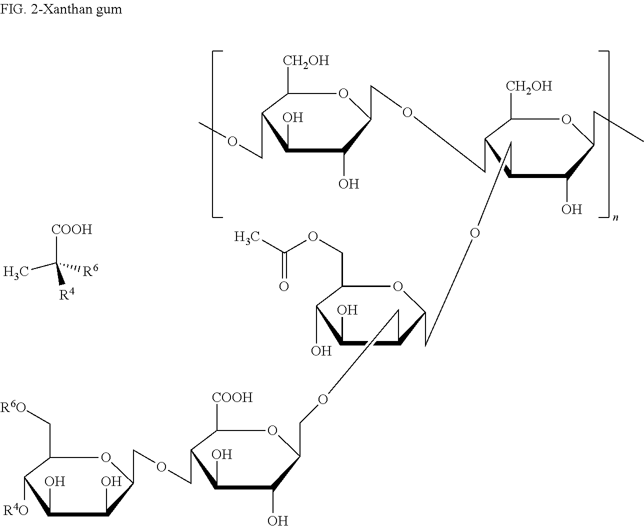

In general, xanthan gum is a substance made by fermenting bacteria with sugars. It is an additive found in both foods and medicines. As a food additive, this substance is utilized either as a thickener or stabilizer. This compound has a variety of uses in medicine, such as in the treatment of diabetes, cholesterol and dry mouth,

Specifically, xanthan gum is a polysaccharide secreted by the bacterium Xanthomonas campestris. It's known uses, prior to the invention, is as a food additive and rheology modifier, commonly used as a food thickening agent (in salad dressings, for example) and a stabilizer (in cosmetic products, for example, to prevent ingredients from separating). As seen in FIG. 1, it is composed of pentasaccharide repeat units, comprising glucose, mannose, and glucuronic acid in the molar ratio 2:2:1. It is produced by the fermentation of glucose, sucrose, or lactose. After a fermentation period, the polysaccharide is precipitated from a growth medium with isopropyl alcohol, dried, and ground into a fine powder. Later, it is added to a liquid medium to form the gum.

### Guar Gum

Chemically, guar gum is a polysaccharide composed of the sugars galactose and mannose. FIG. 3 show that the backbone is a linear chain of β1,4-linked mannose residues to which galactose residues are 1,6-linked at every second mannose, forming short side-branches.

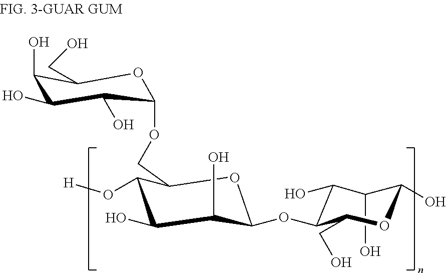

In water, guar gum is nonionic and hydrocolloidal. It is not affected by ionic strength or pH, but will degrade at extreme pH and temperature (e.g. pH 3 at 50° C.). It remains stable in solution over pH range 5-7. Strong acids cause hydrolysis and loss of viscosity, and alkalies in strong concentration also tend to reduce viscosity. It is insoluble in most hydrocarbon solvents. The viscosity attained is dependent on time, temperature, concentration, pH, rate of agitation and practical size of the powdered gum used. The lower the temperature lower the rate at which viscosity increases and the lower the final viscosity. Above 80° the final viscosity is slightly reduced. The finer guar powders swells more rapidly than coarse powdered gum. Guar gum has almost eight times the water-thickening potency of cornstarch—only a very small quantity is needed for producing sufficient viscosity. Thus, it can be used in various multiphase formulations: as an emulsifier because it helps to prevent oil droplets from coalescing, and/or as a stabilizer because it helps to prevent solid particles from settling.

### Cyclodextrin

Cyclodextrins are a group of structurally related natural products formed during bacterial digestion of cellulose. These cyclic oligosaccharides consist of (α-1,4)-linked α-D-glucopyranose units and contain a somewhat lipophilic central cavity and a hydrophilic outer surface. Due to the chair conformation of the glucopyranose units, the cyclodextrins are shaped like a truncated cone rather than perfect cylinders. The hydroxyl functions are orientated to the cone exterior with the primary hydroxyl groups of the sugar residues at the narrow edge of the cone and the secondary hydroxyl groups at the wider edge. The central cavity is lined by the skeletal carbons and ethereal oxygens of the glucose residues, which gives it a lipophilic character. The polarity of the cavity has been estimated to be similar to that of an aqueous ethanolic solution.

The natural α-, β- and γ-cyclodextrin (αCD, βCD and γCD) consist of six, seven, and eight glucopyranose units, respectively. The natural cyclodextrins, in particular βCD, are of limited aqueous solubility meaning that complexes resulting from interaction of lipophiles with these cyclodextrin can be of limited solubility resulting in precipitation of solid cyclodextrin complexes from water and other aqueous systems. In fact, the aqueous solubility of the natural cyclodextrins is much lower than that of comparable acyclic saccharides. This is thought to be due to relatively strong intermolecular hydrogen bonding in the crystal state. Substitution of any of the hydrogen bond forming hydroxyl groups, even by lipophilic methoxy functions, results in dramatic improvement in their aqueous solubility. Water-soluble cyclodextrin derivatives of commercial interest include the hydroxypropyl derivatives of βCD and γCD, the randomly methylated β-cyclodextrin (RMβCD), and sulfobutylether β-cyclodextrin sodium salt (SBEβCD).

FIG. 4 and Table 1 were taken from an article entitled “Cyclodextrins” (A. Magnúsdóttir, M. Másson and T. Loftsson, J. Incl. Phenom. Macroc. Chem. 44, 213-218, 2002).

TABLE 1
Water solubility of cyclodextrins

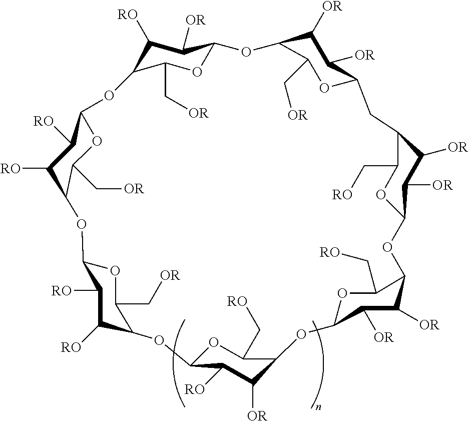

| | | | | | Solubility in |
|---|---|---|---|---|---|
| | | | | MW^{b} | water^{c} |
| Cyclodextrin | n | R = H or | Subst. | (Da) | (mg/L) |
| α-Cyclodextrin (αCD) | 0 | —H | 0 | 972 | 145 |
| β-Cyclodextrin (βCD) | 1 | —H | 0 | 1135 | 18.5 |
| 2-Hydroxypropyl-β-cyclodextrin | 1 | —CH₂CHOHCH₃ | 0.65 | 1400 | >600 |
| (HPβCD; Kleptose ® HPB) | | | | | |
| Sulfobutylether β-cyclodextrin sodium | 1 | —(CH₂)₄SO₃⁻ | 0.9 | 2163 | >500 |
| salt (SBEβCD; Captisol ®) | | Na⁺ | | | |
| Randomly methylated β-cyclodextrin | 1 | —CH₃ | 1.8 | 1312 | >500 |
| (RMβCD) | | | | | |
| γ-Cyclodextrin (γCD) | 2 | —H | 0 | 1297 | 232 |
| 2-Hydroxypropyl-γ-cyclodextrin | 2 | —CH₂CHOHCH₃ | 0.6 | 1576 | >500 |
| (HPγCD) | | | | | |

^{a}Average number of substituents per glucose repeat unit;.
^{b}MW: Molecular weight;
^{c}Solubility in pure water at approx. 25° C.

Cyclodextrins create highly concentrated and water-soluble granules. Cyclodextrins are circular structures of sugar molecules that are known to absorb other compounds into their center. They form inclusion complexes with poorly water-soluble compounds. Acting like a molecule magnet, cyclodextrins absorb other molecules and assume their properties. These molecules can absorb up to 60% of their weight in alcohol while remaining in powdered form. It isn't until you mix them with water that they dissolve.

Experiments with THC-cyclodextrin compounds increase THC water solubility by nearly 1000 times. For this reason in preferred embodiments, the emulsifying agent is a combination of at least two different emulsifying agents with at least one being cyclodextrin and the other emulsifying agent selected from the group consisting of xanthan gum, guar gum, lecithin, carrageen, monoglycerides, natural emulsifiers and organic emulsifiers that are safe for ingestion by humans.

It is noted that cyclodextrin is very expensive and some versions even cause unwanted side effects when ingested. In the invention a lesser amount of cyclodextrin is used in combination with other emulsifiers that are less costly to provide the same or better solubility results. This provides an economic solution to using a lesser amount of cyclodextrin with the benefits at lower cost

Lecithin

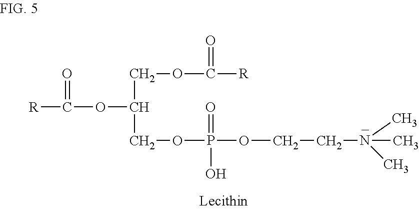

Lecithins are used in the invention as emulsifiers. They are surface-active; simultaneous hydrophilic (water-loving) and hydrophobic (water-repelling) properties enable lecithins to make stable blends of materials that otherwise do not mix easily and tend to separate.

Lecithin is a generic term to designate any group of yellow-brownish fatty substances occurring in animal and plant tissues, which are amphiphilic—they attract both water and fatty substances (and so are both hydrophilic and lipophilic). Lecithins are generally used for smoothing food textures, dissolving powders (emulsifying), homogenizing liquid mixtures, and repelling sticking materials. Lecithins are composed of phosphoric acid with choline, glycerol or other fatty acids usually glycolipids or triglyceride. Glycerophospholipids in lecithin include phosphatidylcholine, phosphatidylethanolamine, phosphatidylinositol, phosphatidylserine, and phosphatidic acid.

When added to cannabis coconut oil lecithin increases absorption of THC and other cannabinoids into the cell membranes and speeds up the process.

### Carrageen

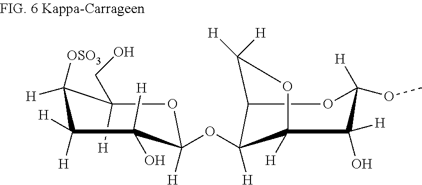

Carrageens are a family of linear sulphated polysaccharides that are extracted from red edible seaweeds. They are widely used in the food industry, for their gelling, thickening, and stabilizing properties. Their main application is in dairy and meat products, due to their strong binding to food proteins. There are three main varieties of carrageenan, which differ in their degree of sulphation. Kappa-carrageenan has one sulphate group per disaccharide, Iota-carrageenan has two, and Lambda-carrageenan has three.

### Monoglycerides

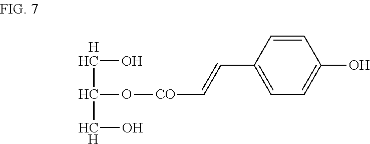

Monoglycerides are a class of glycerides which are composed of a molecule of glycerol linked to a fatty acid via an ester bond.^{[1]} As glycerol contains both primary and secondary alcohol groups two different types of monoglycerides may be formed; 1-monoacylglycerols where the fatty acid is attached to a primary alcohol, or a 2-monoacylglycerols where the fatty acid is attached to the secondary alcohol.

Monoglycerides are primarily used as surfactants, usually in the form of emulsifiers. Together with diglycerides, monoglycerides are commonly added to commercial food products in small quantities which helps to prevent mixtures of oils and water from separating.

### Base Oil

The base oil is preferably in the range of 0.1% to 40% of the emulsification.

The base oil is preferably selected from the group consisting of vegetable glycerine, coconut oil and any oil high in saturated fats. Nut oils are also used in the invention process. The nut oils are selected from the group consisting of almond oil, avocado oil, canola oil, coconut oil, corn oil, cottonseed oil, grapeseed oil, hazelnut oil, olive oil, extra virgin olive oil, palm oil, peanut oil, palm seed oil, pumpkin seed oil, safflower oil, sesame oil, soy oil, sunflower oil, vegetable oil and walnut oil.

### Aqueous Vehicle

The aqueous vehicle is selected from the group consisting of coconut water, fruit juice, milk and water. The aqueous vehicle is in the range of 60% to 99.9% of the emulsification. The preferred vehicle is coconut water.

In the emulsification the base oil:aqueous vehicle ratio is between 1 to 10 grams of base oil per 2 ounces of the emulsification.

### Cannabis Oil

The cannabis oil used in the invention is in a pure state. This is important sine the intended end use of the products of the invention are to be ingested by humans for medical or recreational use, where permitted.

The cannabis oil used can be extracted from the marijuana plant by CO2 extraction, water extraction, butane extraction and extraction methods that leave a zero testing for residuals. Representative structures of the cannabis oil are illustrated in FIG. 1.

The cannabis oil used in the invention is selected from the group consisting of tetrahydrocanniabinol (THC), cannabidiol (CBD) and other cannabinoid oils isolated from the marijuana plant.

The cannabis oil in the emulsification is in the range of 5 mg to 30 mg per 2 ounces of the emulsification.

Caffeine is present in the emulsification in the range of 10 to 300 mg per 2 ounces of the emulsification. The caffeine can be in anhydrous form.

### Method of Making the Emulsifications

The method to produce the invention emulsifications, include first heating a base oil, preferably, extra virgin organic coconut oil to between 120 to 220 degrees F. Pure extracted cannabis oil is added to the heated mixture. In a high speed blender (or similar machine) an aqueous vehicle, preferably coconut water, is added to the coconut fat (oil) to insure emulsification. While blending the heated mixture, adding at least one emulsifying agent in the amount of 0.15% and 2% of the total volume of finished product, to the heated oil to create a mixture. Percentages's used herein are on a dry weight basis and are based on the total volume of the finished product. The blender is run at high speed for between 30 seconds and 2 minutes before adding the anhydrous caffeine in amounts ranging from 10-300 mg. Alternatively, the caffeine can be added prior to adding the emulsifying agent or at the same time. The resulting cannabinoid emulsification is bio-available, highly metabolizable and fast acting when ingested by the user. The resulting emulsification is used to produce a line of THC and caffeine infused emulsifications. The invention process makes the cannabis oil more bioavailable by making the oleo molecule water soluble. Thus, upon ingestion, making it fast acting, taking effect in as little as 15 minutes.

Other variations include various doses of cannabis oil in the range of 5 to 30 mg and different flavor profiles including lime, pomegranate, orange, lemon and others; and different serving sizes between 1 and 64 oz

The disclosure is further described with the help of the following examples. These examples, however, should not be construed to limit the scope of the disclosure.

### Example 1

A cannabis infused chocolate is provided that is bioavailable and delivers fast acting effects of the cannabis when ingested. The method of making such includes use of 5 to 10 oz of a base oil of either vegetable glycerine or coconut oil. A high quality liquor such as cognac or whiskey can be added but is optional. The base oil liquid is heated to between 120 to 220° F. The cannabis oil extract is added equal to 110 to 1120 mg THC. The emulsifiers are added next, generally in the following amounts 0.5% lecithin, 0.15% xanthan gum, 0.1% cyclodextrin. The emulsifiers can be used individually or in combination. The hot mixture is blended in a high speed blender or other machine, run on high speed for 2 minutes. The mixture is allowed to cool to room temperature.

After the mixture has cooled 10 lb of melted chocolate is added and allowed to temper before depositing in a mold than cooling to 55° F.

### Example 2

Several experiments were run using several different emulsifiers and combinations of different coconut oils: solid and liquid (MCT). (Note: liquid MCT is coconut oil that has medium chain triglyceride). Guar gum, lecithin, and cyclodextrin were tested as emulsifying agents and provided good results. However xantham gum was the most effective and provided the best emulsification of the oil and water, at the lowest viscosity

The emulsification process that was determined the best had the added effect of making the THC more bioavailable by making the oleo molecule water soluble. This had another added effect of making it fast acting, taking effect in as little as 15 minutes

The method used to produce the invention emulsifications, included first heating extra virgin organic coconut oil to between 120 to 220 degrees F. CO2 extracted cannabis oil is added. In a high speed blender (or similar machine) coconut water is added to the coconut fat (oil) to insure emulsification. While blending, xantham gum powder is added in an amount between 0.15% and 0.45% of the total volume of finished product. %'s used herein are on a dry weight basis and are based on the total volume of the finished product. The blender is run at high speed for between 30 seconds and 2. The resulting emulsification is used to produce a variety of cannabis infused products. The invention process makes the THC more bioavailable by making the oleo molecule water soluble. Thus, upon ingestion, making it fast acting, taking effect in as little as 15 minutes.

### Example 3

A variety of cannabis infused products were prepared and tested in a random study group of 40 individuals. The products tested included cannabis infused sugar, a cannaboid/caffeine emulsification and a cannabis infused elixir and are summarized in the tables below. The products in Table 2 and 4 are the subject of a co-pending patent application by the same inventor entitled “Cannabis Infused Sweeteners and Elixirs.”; and the subject of a co-pending patent application by the same inventor entitled “Method of Making Cannabis Oil Hydrophilic Using Emulsifiers and Related Cannabinoid Compositions” is relevant to making these products, all of which is incorporated herein by reference.

Each of the 40 individuals tested one of the products from Tables 2, 3 and 4. The breakdown of products tested was 10% (4 people) of the cannabinoid/caffeine emulsification; 20% (8 people) of cannabinoid elixirs and 70% (28 people) of the cannabis infused sugar.

TABLE 2
Cannabis infused sugar (Serving size 1 tsp)

| | Sugar Product | | | | |
|---|---|---|---|---|---|
| | #1 | #2 | #3 | #4 | #5* |
| Cannabis Oil | 20 | 40 | 20 | 40 | 40 |
| (mg/tsp) | | | | | |
| Sugar (lbs) | 5 | 3 | 10 | 3 | 3 |
| Alcohol (oz) | 20 | 12 | 40 | 12 | 6 |
| Lecithin (%)** | 2 | 2 | 2 | 1 | 0 |
| Cyclodextrine(%)** | 0 | 0 | 0.03 | 0.12 | 0.25 |

*The sugar used in this sample was maple sugar.
**% of final product.

TABLE 3
Cannabinoid/caffeine emulsification (Serving size 2 oz)

| | Amount in |
|---|---|
| Component | emulsification |
| THC (per serving) | 10 mg |
| Coconut Fat (SOLID) | 2.75% |
| MCT | 0.65% |
| Coconut Water | 96% |
| Cyclodextrin | 0.12% |
| Xanthan Gum | 0.12% |
| Caffeine | 1000 mg |

*Lime and coconut extract were added for flavor

TABLE 4
Cannabinoid elixirs (flavored syrups) (Serving size 1 oz)

| | Amount in |
|---|---|
| Component | emulsification |
| THC | 10 mg/oz |
| Flavored syrup | 2.75% |
| Cyclodextrin | 0.16% |
| Xanthan Gum | 0.12% |
| Cannabis Oil | 0.04% |

Participants in the study were asked a series of questions, the results of which are summarized in the tables below. Q1. How long until you experienced an initial onset of effect after ingestion? The results are in Table 5. In all three products tested the onset of the cannabis effects were less than 15-20 min.

TABLE 5
Results for Onset of Effect

| | Time | | | | | |
|---|---|---|---|---|---|---|
| | >10 | | 15-20 | 20-30 | 30-40 | |
| | min | 10-15 min | min | min | min | <40 min |
| Sugar | 10.71% | 28.57% | 32% | 7.14% | 7.14% | 14.29% |
| Emulsification | 0 | 50% | 50% | 0 | 0 | 0 |
| Elixir | 12.4% | 25% | 25% | 25% | 12.5% | 0 |

Q2. On a scale of 1 to 5, the participants were asked to describe the strength of the initial onset experience after ingestion. A majority of respondents said the effects were mild to moderate. The results are in Table 6.

TABLE 6
Results for Strength

| | 1 No | 2 Very | | | |
|---|---|---|---|---|---|
| Time | effect | Mild | 3 Mild | 4 Moderate | 5 Strong |
| Sugar | 3.57% | 3.57% | 39.29%% | 46.43% | 7.14% |
| Emulsification | 0 | 0 | 50% | 50% | 0 |
| Elixir | 0 | 25% | 50% | 12.5% | 12.5% |

Q3. Compared to other cannabis edibles, the participants were asked how they would characterize the rapidity of the onset of the products they tested. The respondents were comparing the invention products to other products they ingested including gummy bears, brownies and baked goods containing cannabis. The results are in Table 7.

TABLE 7
Results for Comparsion to Other Cannabis Edibles

| | | 2 | | 4 | |
|---|---|---|---|---|---|
| | 1 Much | Somewhat | 3 No | Somewhat | 5 Much |
| Time | Slower | slower | difference | faster | Faster |
| Sugar | 3.85% | 3.85% | 0 | 34.62% | 57.69% |
| Emulsification | 0 | 0 | 0 | 75% | 25% |
| Elixir | 0 | 0 | 0 | 25% | 75% |

In sum, in all embodiments, i.e. the cannabis infused sugar, emulsification and elixir 92.7% to 100% said that the invention products acted faster than other cannabis edibles.

The cannabinoid and caffeine emulsifications of the invention provide a beneficial experience for people experiencing a variety of ailments: insomnia, muscle aches, anxiety, etc, or are in recovery from surgery, or in chemotherapy. The emulsifications makes the cannabis oil molecules hydrophilic, and thus water soluble, which makes the THC, bioavailable, faster acting, and more highly metabolizable.

Medical marijuana patients are often challenged by the mediums they are offered for consuming cannabis. The water-soluble cannabis of the invention provides them a convenient, and smokeless, alternative to access the cannabinoids they need to alleviate their ailments.

The foregoing description of various and preferred embodiments of the present invention has been provided for purposes of illustration only, and it is understood that numerous modifications, variations and alterations may be made without departing from the scope and spirit of the invention as set forth in the following claims.

1. A cannabinoid emulsification comprising:
at least one emulsifying agent selected from the group consisting of xanthan gum, guar gum, cyclodextrin, lecithin, carrageen, monoglycerides, natural emulsifiers and organic emulsifiers that are safe for ingestion by humans;
an aqueous vehicle selected from the group consisting of coconut water, fruit juice, milk and water;
a base oil;
cannabis oil; and
caffeine; wherein the emulsification modifies said cannabis oil such that it is hydrophilic and soluble in said aqueous vehicle.

2. The emulsification according to claim 1, wherein said cannabinoid emulsification is bio-available, highly metabolizable and fast acting when ingested by the user.

3. (canceled)

4. The emulsification according to claim 1, wherein said base oil is selected from the group consisting of vegetable glycerine, almond oil, avocado oil, canola oil, coconut oil, corn oil, cottonseed oil, grapeseed oil, hazelnut oil, olive oil, extra virgin olive oil, palm oil, peanut oil, palm seed oil, pumpkin seed oil, safflower oil, sesame oil, soy oil, sunflower oil, vegetable oil and walnut oil and any oil high in saturated fats.

5. The emulsification according to claim 1, wherein said cannabis oil is selected from the group consisting of tetrahydrocanniabinol (THC), cannabidiol (CBD) and other cannabinoid oils isolated from the marijuana plant.

6. (canceled)

7. The emulsification according to claim 1, wherein said emulsifying agent is a combination of at least two emulsifying agents.

8. The emulsification according to claim 7, wherein said emulsifying agents are cyclodextrin and one emulsifying agent selected from the group consisting of xanthan gum, guar gum, lecithin, carrageen, monoglycerides, natural emulsifiers and organic emulsifiers that are safe for ingestion by humans

9. The emulsification according to claim 1, wherein the cannabis oil is in the range of 5 mg to 30 mg per 2 ounces of the emulsification.

10. The emulsification according to claim 1, where the caffeine is in the range of 10 to 200 mg per 2 ounces of the emulsification.

11. The emulsification according to claim 1, where the emulsifying agent is in the range of 0.15% and 2% of the total volume of the emulsification.

12. The emulsification according to claim 1, where the aqueous vehicle is in the range of 60% to 99.9% of the emulsification.

13. The emulsification according to claim 1, where the base oil is in the range of 0.1% to 40% of the emulsification.

14. The emulsification according to claim 1 wherein said caffeine is anhydrous caffeine.

15. The emulsification according to claim 1, wherein said base oil:aqueous vehicle ratio is between 1 to 10 grams of base oil per 2 ounces of the emulsification.

16. A method for making cannabinoid emulsification comprising the steps of:
heating coconut oil to between 120 to 220 degrees F.;
adding at least one emulsifying agent selected from the group consisting of xanthan gum, guar gum, cyclodextrin, lecithin, carrageen, monoglycerides, natural emulsifiers and organic emulsifiers that are safe for ingestion by humans, caffeine and cannabis oil to an aqueous vehicle;
adding said aqueous vehicle to the heated coconut oil to create a mixture;
blending said mixture in a high speed machine, while holding the temperature between 120 to 220 degrees F. to emulsify the mixture;
and adding caffeine to said mixture; wherein the emulsification modifies said cannabis oil such that it is hydrophilic and soluable in said aqueous vehicle.

17. (canceled)

18. The method according to claim 16, wherein said emulsifying agent is added in an amount between 0.15% and 2% of the total volume of said mixture.

19. The method according to claim 16, wherein the mixture is blended at high speed for between 30 seconds and 2 minutes.

20. The method according to claim 16, wherein the cannabinoid emulsification is bio-available, highly metabolizable and fast acting when ingested by the user.

21. A cannabinoid emulsification comprising:
at least one emulsifying agent present in an amount between 0.15% and 2% of the total volume of the emulsification wherein said emulsifying agent is selected from the group consisting of xanthan gum, guar gum, cyclodextrin, lecithin, carrageen, monoglycerides, natural emulsifiers and organic emulsifiers that are safe for ingestion by humans;
60% to 99.9% of an aqueous vehicle selected from the group consisting of coconut water, fruit juice, milk and water;
0.1% to 40% of a base oil;
cannabis oil present in an amount between 5 mg to 30 mg per 2 ounces of the emulsification; and
caffeine present in an amount between 10 to 200 mg per 2 ounces of the emulsification;
wherein the cannabinoid emulsification is bio-available, highly metabolizable and fast acting when ingested by the user.
